(19) United States
(12) Patent Application Publication
(10) Pub. No.: US 2005/0015103 A1
(43) Pub. Date: Jan. 20, 2005

(54) Minimally invasive device

(76) Inventor: Sergey Popov, Beer-Sheva (IL)

Correspondence Address: DANIEL J SWIRSKY, PO BOX 2345, BEIT SHEMESH, 99544, IL

(21) Appl. No.: 10/500,120

(22) PCT Filed: Dec. 23, 2002
(86) PCT No.: PCT/IL02/01029

(30) Foreign Application Priority Data
Dec. 26, 2001 (IL) 147324

(51) Int. Cl. A61B 17/08

(52) U.S. Cl. 606/153

(57) ABSTRACT

A minimal invasive device for minimal traumatic surgery partly introduced into body cavity, assembled through at least two—the first and the second surgical openings in a body cavity wall, passing through said first surgical opening during device operation, and having: An inner unit, an outer unit, and an intermediate part, which is an integral part of one of said units, has a free end, is designed to passing through body cavity wall via said first surgical opening, and having a maximal transverse dimension substantially lesser than a maximal transverse dimension of said inner unit; said intermediate part in assembled operating devise is disposed between said units and inside said first surgical opening, Said inner unit designed to inserting into body cavity and withdrawing therefrom through said second surgical opening and having maximal transverse dimension, which is substantially more than maximal transverse dimension of said first surgical opening minimally needed for inserting therethrough said intermediate part thereby eliminating the insertion of said inner unit into body cavity and withdrawing therefrom through said first surgical opening, A coupling means partly disposed on said intermediate part and adapted to operative connecting and disconnecting said inner unit and said outer unit while said inner unit is disposed inside body cavity.

### FIELD OF THE INVENTION

The present invention generally relates to the minimally invasive device for minimal traumatic surgery executed in patient's body cavity, and more particularly, for abdominal and thoracic surgery. The invention also includes the methods of minimally traumatic surgical operations and the instruments for their execution.

### BACKGROUND OF THE INVENTION

Traditional surgical instrument for minimal invasive surgery (MIS) includes a handle at its proximal part, a housing, and a functional head at its distal end. This head is inserted into patient's body cavity through a port mounted in body cavity wall by trocar. Main requirement of MIS lies in the minimization of the surgical opening dimensions needed for mounting the mentioned port. These dimensions are predetermined with the dimensions of the instrument head. Therefore, the noted MIS requirement also demands the minimization of instrument head dimensions that needs significant developments and investments and not always is achieved. Moreover, the noted MIS requirement interferes with using in MIS the surgical instruments of open surgery. This, to a large extent, withstands MIS spreading.

Endoscopic Surgical Anastomosis Stapling Instrument is disclosed by U.S. Pat. No. 5,333,773. The instrument is designed for bowel circular anastomosis and includes a suture head with a staples' cartridge immovably connected to instrument housing and detachable anvil disposed distally of the staples' cartridge. Diameters of the cartridge and the anvil are equal. The disadvantage of the instrument lies in relatively large dimensions of its part (cartridge) inserted into body cavity through trocar port. This demands relatively large surgical opening in body cavity wall. The necessity of using the suture head of larger diameter inevitably leads to the increase of needed surgical opening.

Instrument for Circular Surgical Stapling is disclosed by U.S. Pat. No. 4,603,693. The instrument has a disposable suture head detachably connected to the housing and consisting of a staples' cartridge and an anvil. The staples' cartridge is connected to the housing before inserting into body cavity. It means, that the instrument, in the same manner as previous instrument (U.S. Pat. No. 5,333,773), demands relatively large surgical opening in body cavity wall determined with the dimensions of the staples' cartridge. Moreover, the anvil does not have the anvil shaft that eliminates inserting separated anvil into patient bowel and demands complete assembling the suture head before its operation

Linear Suturing Apparatuses for use on mesentery, omentum and the like are disclosed by U.S. Pat. Nos. 4,671,445 and 5,027,834. In this case, the suture head has long linear staples' cartridge and anvil and, as a consequence, relatively large transverse dimension of suture head. As a result, the application of these apparatuses in MIS is practically impossible.

Method for Performing a Coronary Artery Bypass is disclosed by U.S. Pat. No. 6,167,889. The surgical operations are performed on closed thorax through a plurality of intercostal ports. The disadvantage of this method is impossibility of using relatively large instruments because of limited dimensions of intercostal gaps. Moreover, the control of internal organs positions and their fixation is very inconvenient and demands many special instruments and ports. This restricts the surgical possibilities and leads to degradation in the quality of surgical procedure.

The common disadvantage of all the above noted patents is the necessity of using traditional trocar devices for ports mounting, for instance disclosed by U.S. Pat. No. 5,690,663. These trocar devices include a port unit and an obturator with sharp distal end for penetrating into body cavity from the outside. Typically, there is a danger of internal organs injury by the obturator distal sharp end at the moment of its entering body cavity. Developed protectors of the obturator sharp end (see the noted U.S. Pat. No. 5,690,663) increase device cost and allow only partial solution of this problem. The mentioned injury danger demands the concentration of surgeon attention and its significant nerves tension.

An Endoscopic Surgical Sealing Device is disclosed by U.S. Pat. No. 5,366,478. The device enables the inserting into body cavity either surgeon's hand or relatively large surgical instrument through a hand port. The disadvantage of such hand port applying is the impossibility of hand port use according to its direct designation during its use as a port for surgical instrument. Using the hand port for surgical instrument insertion does not meet the noted main MIS requirement to minimize the surgical opening. The same relates to another embodiment of this patent with obviously enlarged surgical opening and port for inserting the surgical instruments of various dimensions.

### SUMMARY OF THE INVENTION

The objective of the present invention is the minimization of surgical openings dimensions in body cavity wall for inserting surgical instruments into body cavity.

Another objective is the minimization of surgical openings number in body cavity.

Another objective is the extension of applying in MIS the surgical instruments of relatively large dimensions.

Another objective is using in MIS main components of surgical instruments used in open surgery.

Another objective is decreasing needed investment for the development of needed surgical instrument set.

Another objective is improving the convenience of surgical operations execution and decreasing the surgeon nerves tension.

Another objective is substantial extension of surgical possibilities and rise in the quality of surgical operations.

Another objective is the decrease of total instruments number needed for surgical operations execution.

Another objective is reducing the total cost of surgical instruments set needed for surgical procedure performance.

Another objective is significant lowering the possibility of patient's internal organ injury during surgical procedure.

Another objective is imparting universal properties to the surgical instruments allowing its use both in MIS and in open surgery.

The above noted objectives are accomplished with a minimal invasive device for minimal traumatic surgery, assembled through at least two—the first and the second surgical openings in a body cavity wall, passing through the first surgical opening during device operation and having an inner unit, an outer unit, and an intermediate part. The latter is an integral part of one of mentioned units and has a free end designed to passing through body cavity wall via the first surgical opening. The maximal transverse dimension of the intermediate part and its free end is substantially lesser than one of the inner unit. The inner unit is designed for inserting into body cavity and withdrawing therefrom through the second surgical opening and has the maximal transverse dimension substantially more than one of the first surgical opening minimally needed for inserting therethrough the intermediate part. As a result, the insertion of the inner unit into body cavity and withdrawing therefrom through the first surgical opening is impossible. The device also includes a coupling means partly disposed on the intermediate part and having fast acting connectors adapted to operative connecting and disconnecting the inner and outer units while the inner unit is disposed inside body cavity.

The method of preparation and execution of minimally traumatic surgical operations with the above noted minimally invasive device comprises: forming the first and the second surgical openings in body cavity wall, inserting the inner unit into body cavity through the second surgical opening, leading the free end of the intermediate part through body cavity wall via the first surgical opening, connecting the inner and outer units by the fast acting connectors with forming an operational assembly, execution of surgical operations at least by the mentioned operational assembly, disconnecting the inner and outer units after executing needed surgical operations, and withdrawing the device components through the same openings through which they have been inserted: the intermediate part through the first surgical opening and the inner unit from body cavity through the second surgical opening. The second surgical opening can be embodied as a hand port or some enlarged trocar port. The first surgical opening can be carried out as small trocar port with inner diameter of the order of 6-13 mm.

Thus, only small first surgical opening is necessary for inserting surgical instrument into body cavity. In spite of using one some enlarged second surgical opening, substantial decrease of patient's traumatization is achieved since, as a rule, many surgical instruments and, correspondently many first surgical openings, are used in MIS (see, for instance U.S. Pat. No. 6,167,889, FIG. 11). The present invention allows the use of the inner units of various dimensions and functional designations with the same one outer unit thereby decreasing needed number of the first surgical openings. This also decreases patient's traumatization, reduces needed instrument number and its cost. Moreover, the first surgical opening does not restricts the dimensions of instrument's functional head (inner unit), which is inserted into body cavity in separated form through relatively large second surgical opening. It means, that the present invention substantially extents the surgical possibilities of MIS due to applying in MIS significantly enlarged functional heads, including used in open surgery. The latter enables the application in MIS already developed and well-checked designs of open surgery thereby decreasing the expected investment for R&D. The present invention allows the improvement of operation convenience and the rise in quality of surgical operations due to the possibility of immediate participation of surgeon's hand inserted through the second surgical opening in surgical manipulation inside body cavity. The latter is especially important for thoracal surgery and, in this case, the present invention provides for the carrying out the second surgical opening, which begins in epigastrium.

In version embodiment, the present invention provides for the execution of the first surgical openings from the inside of body cavity by simplified obturatorless trocar device previously inserted into body cavity through the second surgical opening. In another version, the first surgical opening is executed without trocar device by means of sharp penetrating tip disposed on the free end of device intermediate part. These versions additionally decrease the cost of surgical instruments cost, enhance the operation safety eliminating the danger of internal organs injury by trocar sharp penetrating end, and decrease needed concentration of surgeon's attention caused with this danger.

In version embodiment, the minimally invasive device presents a circular stapler for bowel circular anastomosis, wherein the anvil is disposed distally of the staples' cartridge unit. The anvil unit has the anvil and an anvil shaft. There are also a drive means including a control rod connecting the actuating means and the anvil shaft as well as a drive means connector including an anvil shaft engagement member and a control rod engagement member. The cartridge unit is secured to a body of device intermediate part by units bodies connector and includes a connector lock means eliminating the disconnection of the drive means connector at least during tissue stapling. The anvil and cartridge units form the stapler's inner unit inserted into body cavity and withdrawn therefrom through the second surgical opening. The distal free end of the control rod presents the free end of stapler's intermediate part inserted into body cavity through the first surgical opening. The drive means connector and units bodies connector are fast acting connectors allowing operative connecting and disconnecting the stapler's intermediate part and inner unit when the latter is disposed in body cavity. The mentioned lock means provides reliable stapler operation during tissue suturing. The stapler provides the possibility of the operation according to the above noted method of the present invention with all the noted advantages.

Note, proposed minimally invasive devices are universal instruments allowing their use both in present proposed MIS and in open surgery, as well as in usual MIS as a combination of traditional miniature functional head of MIS with the outer part of proposed instrument.

### BRIEF DESCRIPTION OF THE DRAWINGS

FIG. 1 shows the assembled circular stapler for bowel circular anastomosis.

FIG. 2 shows the cross-section of all the components of the circular stapler.

FIG. 3 shows the cross-section of the circular stapler after removing an anvil unit.

FIG. 4 shows the distal part of the circular stapler with extended anvil.

FIG. 5 shows the circular stapler at the moment immediately before tissue suturing start.

FIG. 6 shows the circular stapler at the moment of tissue suturing end.

FIG. 7 shows the circular stapler in the state of its insertion into body cavity.

FIG. 8 shows the circular stapler with sutured bowel parts secured to stapler.

FIG. 9 shows the circular stapler and sutured bowel parts at the moment of suturing start.

FIG. 10 shows the circular stapler and sutured bowel parts at the moment of suturing end.

FIG. 11 shows obturatorless trocar device with detachable penetrating tip.

FIG. 12 shows the fixation member fotrocar port fixation in body cavity wall.

FIG. 13 shows the piercing the body cavity wall by obturatorless trocar.

FIG. 14 shows the obturatorless trocar at the moment of its going out of body cavity wall.

FIG. 15 shows the trocar port introduced into body cavity wall by obturator less trocar from the inside of body cavity.

FIG. 16 shows the trocar port introduced into body cavity wall by obturatorless trocar from the outside of body cavity.

### DETAILED DESCRIPTION OF THE INVENTION

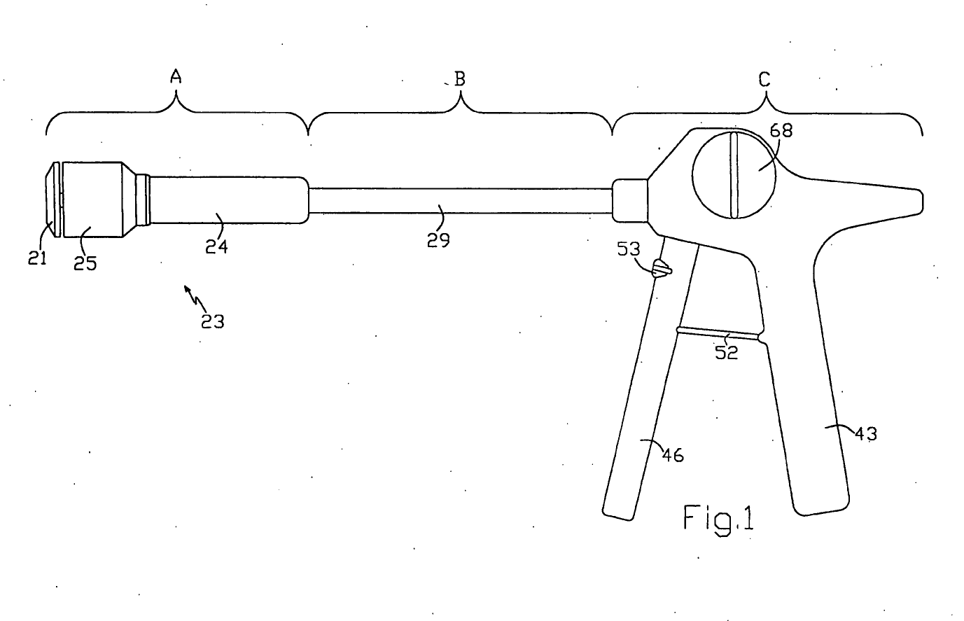

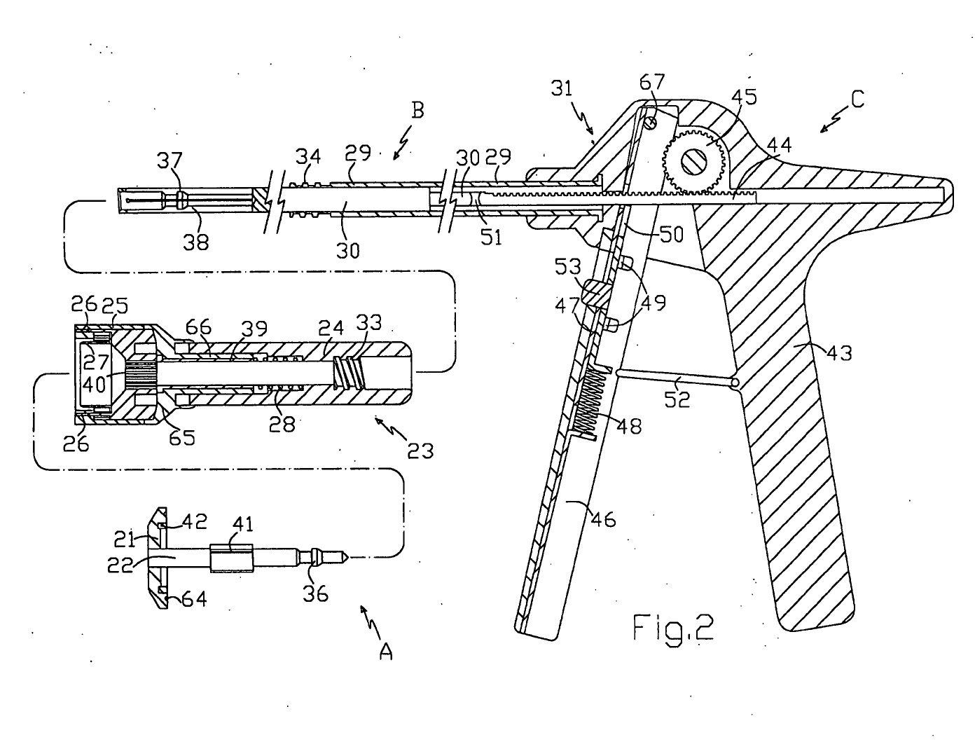

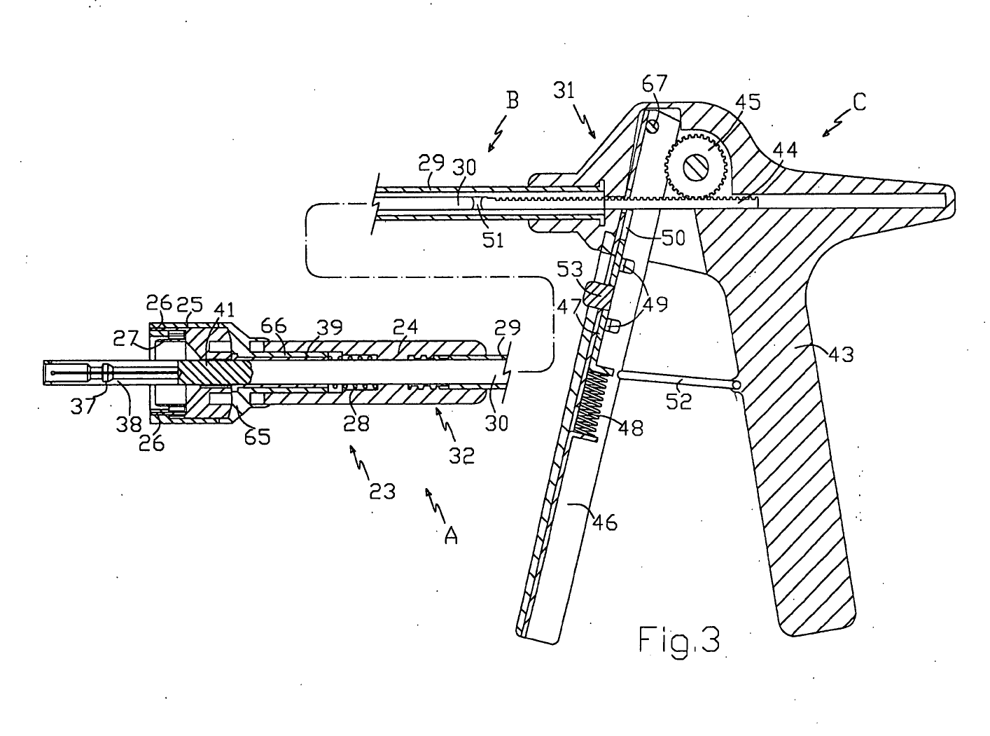

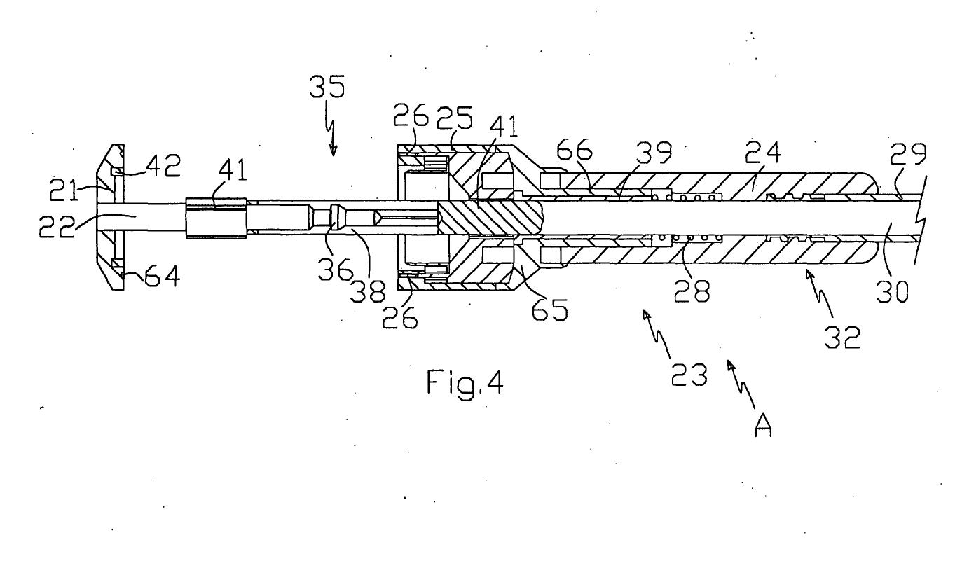

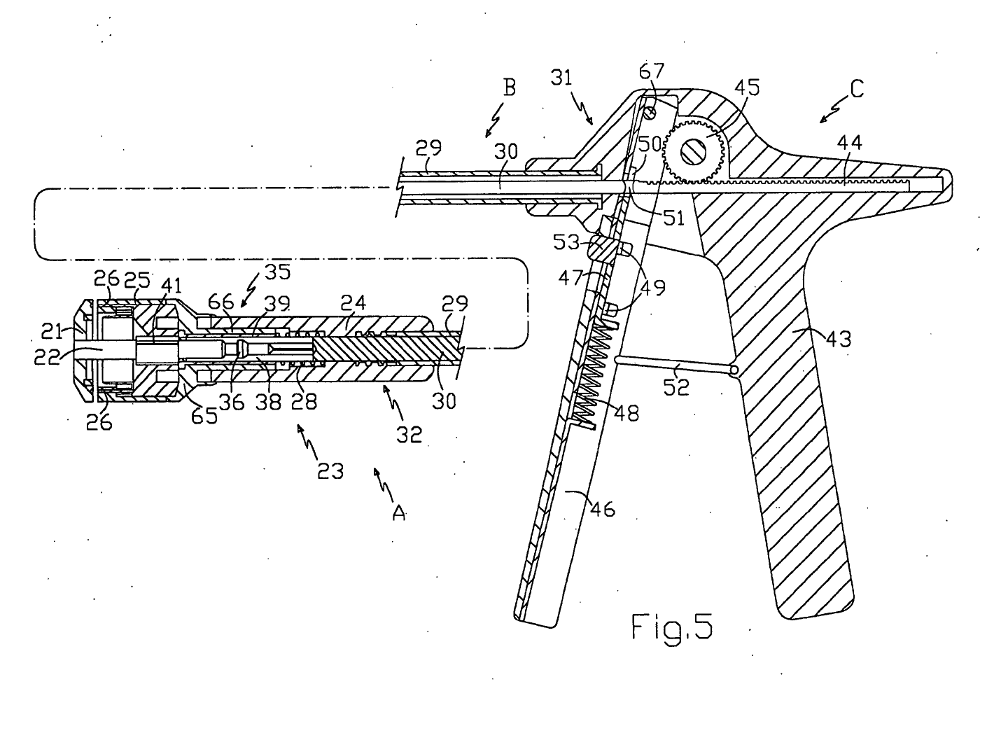

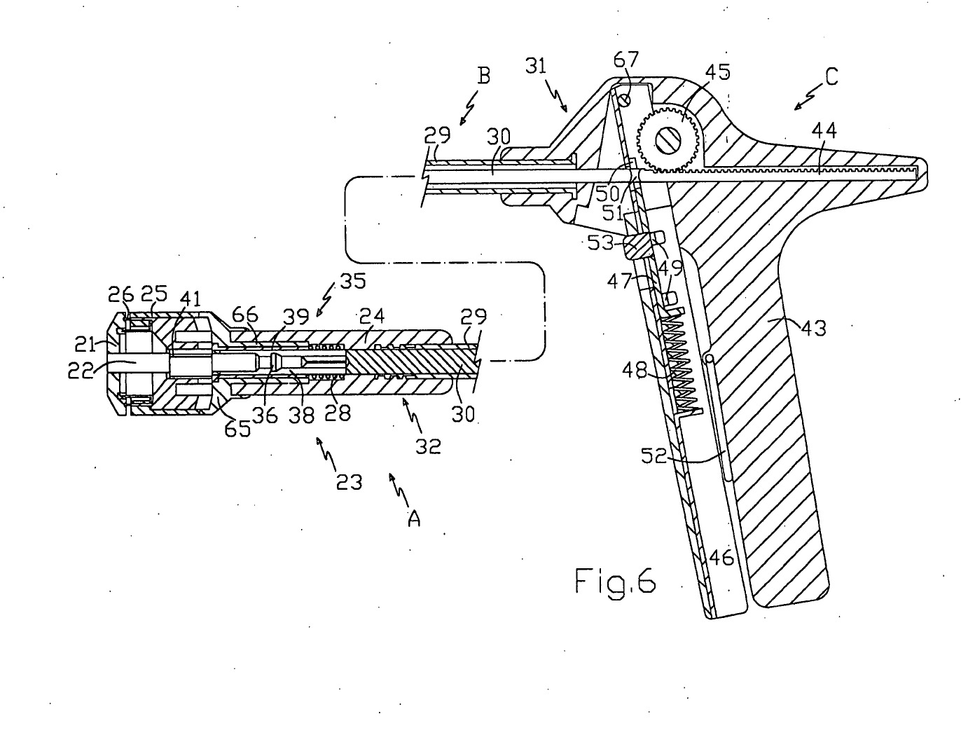

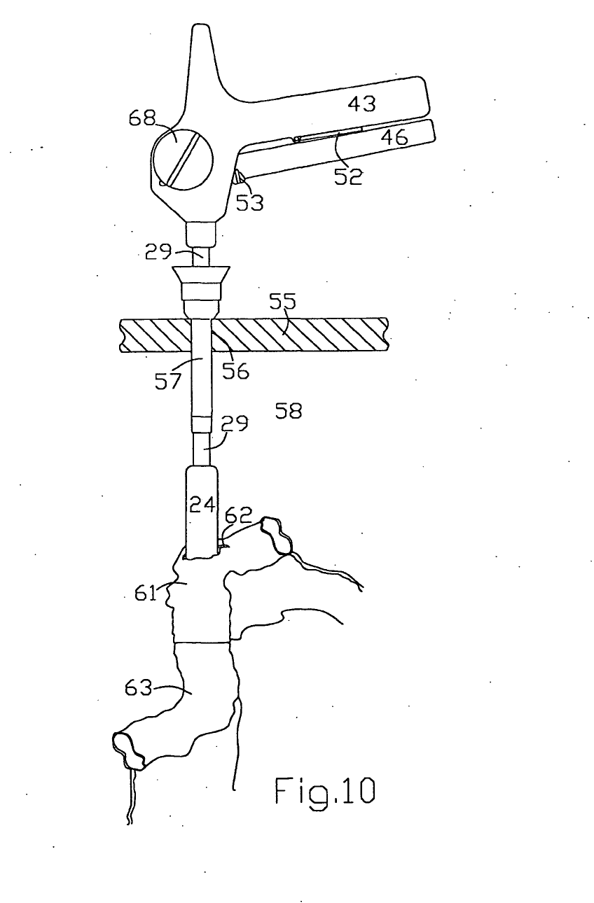

With reference now to the drawings, the minimally invasive device in the form of circular stapler according to the present invention includes an inner unit A, an intermediate part B, and an outer unit C (see FIG. 1). The inner unit A has anvil unit 20 including anvil 21, malleable web 42, staple bending grooves 64, and anvil shaft 22, as well as cartridge unit 23 including inner unit body 24, cartridge carrier 65, cartridge carrier base 66, cartridge 25 having a plurality of staples 26, and circular knife 27 interacting with malleable web 42 during cutting off excessive bowel tissue after bowel parts suturing. In assembled stapler, anvil 21 is disposed distally of cartridge 25. Cartridge 25 is movable in axial direction and underspringed by spring 28 relative to inner unit body 24. The intermediate part B has intermediate part body 29 housing the drive means in the form of control rod 30 connecting actuating means 31 with anvil shaft 22. Cartridge unit 23 can be connected to/disconnected from intermediate part body 29 by units bodies connector 32 in the form of thread 33 on inner unit body 24 and 34 on intermediate part body 29. Anvil shaft 22 can be connected to/disconnected from control rod 30 by drive means connector 35 including an anvil shaft engagement member in the form of circular projection 36 on the proximal free end of anvil shaft 22 and a control rod engagement member in the form of circular recess 37 of collet 38 on the distal free end of control rod 30. Cartridge unit 23 also includes a connector lock means in the form of bushing 39 immovably connected with cartridge carrier base 66 and hampering transverse broadening the collet 38 thereby eliminating the disconnection of drive means connector 35 during tissue stapling (FIGS. 5, 6). There are also means for mutual angular orientation of anvil 21 and cartridge 25 in the form of inner grooves 40 and outer oblong projections 41. Outer unit C includes handle 43 and actuating means 31. The latter comprises a proximal end portion of control rod 30 in the form of rack 44 interacting with toothed wheel 45 manually controlled by wheel handle 68 and designed for control rod retraction to bring together anvil 21 and cartridge 25 and bowel parts before their suturing as well as for anvil extension distally to withdraw drive means connector 35 from locking bushing 39 for the disconnection of connector 35 after bowel part suturing. Outer unit C also includes lever 46 rotatable around axle 67 and designed for transmitting to control rod 30 the force intended for bending staples 26 and thereby immediate suturing bowel parts. Lever 46 is provided with engagement member 47 undespringed by spring 48, sliding in guides 49, and having upper fork 50 for engaging notch 51 of control rod 30 at the moment of bringing together anvil 21 and cartridge 25 immediately before staples' bending and bowel part suturing (FIG. 5). After putting down locking protector 52 (FIG. 6), surgeon brings together handle 43 and lever 46 thereby compressing spring 28 and displacing cartridge 25 proximally. As a result, staples 26 are gone out of cartridge 25 and bent by grooves 64 of anvil 21 thereby suturing bowel parts (FIG. 10). At the same time, knife 27 cuts off excessive bowel tissue. Then, surgeon puts down knob 53 of engagement member 47 thereby disengaging fork 50 and notch 51 and allowing the extension distally of control rod 30 by wheel 45 to provide the possibility of disconnecting the drive means connector 35 (FIG. 4).

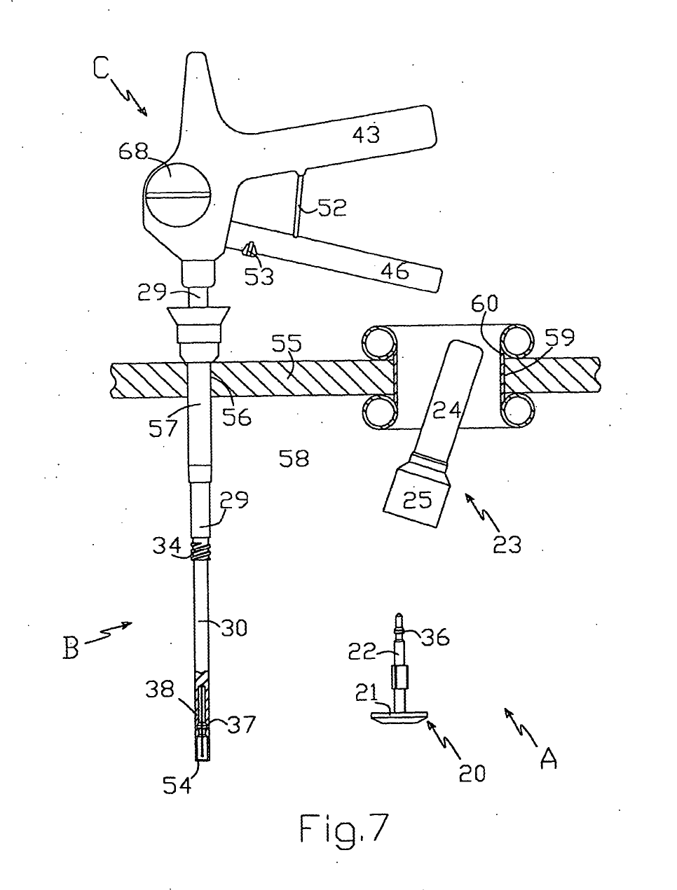

Intermediate part B and, specifically, its body 24, forms an integral part with outer unit C and has distal free end 54 designed for passing through body cavity wall 55 via first surgical opening 56 or via trocar port 57 mounted in first surgical opening 56 (FIG. 7). Maximal transverse dimension of intermediate part B, specifically of its body 29, is substantially lesser than maximal transverse dimension of inner unit, specifically of cartridge 25. Inner unit A including anvil unit 20 and cartridge unit 23 is designed for inserting into body cavity and withdrawing therefrom through second surgical opening 59 or through hand port 60 mounted in second surgical opening 59. Maximal transverse dimension of inner unit A, specifically of cartridge 25, is substantially more than maximal transverse dimension of first surgical opening 26 or inner diameter of trocar port 57 minimally needed for inserting therethrough intermediate part B. As a result, the insertion of inner unit A into body cavity 58 and withdrawing therefrom through first surgical opening 56 or trocar port 57 is impossible. A coupling means of circular stapler including units bodies connector 32 and drive means connector 35 are adapted to operative connecting and disconnecting inner unit A and the block of outer unit—intermediate part while inner unit A is disposed inside body cavity 58.

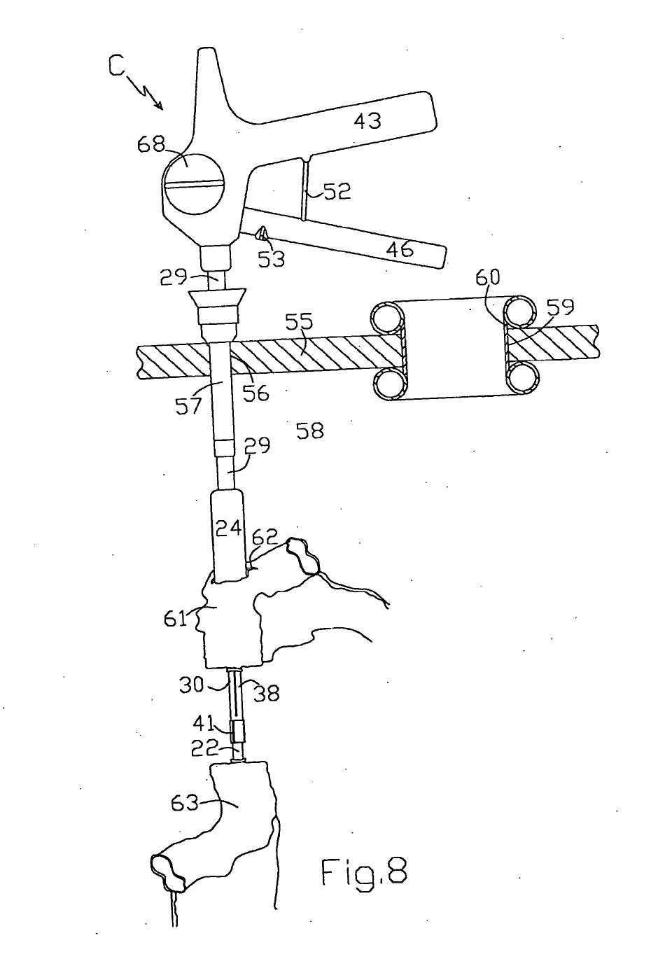

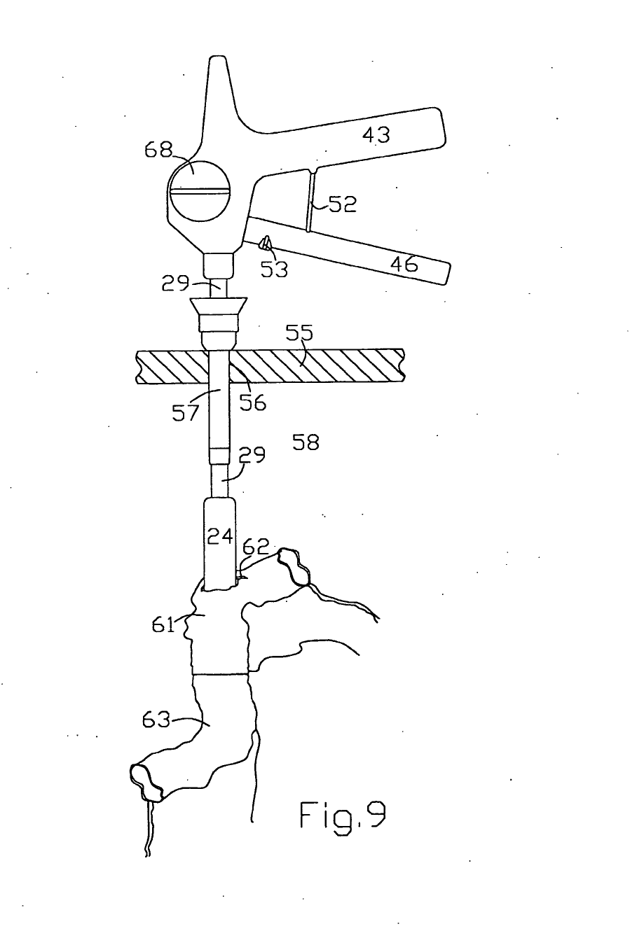

Method of preparation and execution of minimally traumatic surgical operations implemented with above described circular stapler comprises forming first surgical opening 56 in body cavity wall 55 by trocar device and mounting trocar port 57 therein, and forming second surgical opening 59 and mounting hand port 60 therein. First surgical opening 56 has minimally needed maximal transverse dimension substantially lesser than one of second surgical opening 59 and of cartridge 25. The same relates to the relationship of inner diameters of trocar port 57 and hand port 60, respectively. Further, method provides for inserting inner unit A including anvil unit 20 and cartridge unit 23 into body cavity 58 through hand port 60, and distal free end 54 of control rod 30 and distal end of intermediate part body 29 into body cavity 58 through trocar port 57. Then, inner unit A is connected to outer unit C through intermediate part B by coupling members with forming an operation assembly. As applied to the circular stapler, this connection is executed in the following manner: first, cartridge unit 23 is connected to intermediate part body 29 by units bodies connector 32, then cartridge 25 is inserted into bowel part 61 through lateral incision 62 and bowel part 61 is secured to control rod 30 from the distal side of cartridge 25, then anvil 21 is inserted into bowel part 63 and the latter is secured to anvil shaft 22, then anvil unit 20 is connected to control rod 30 by drive means connector 35 (FIG. 8). Further, surgeon executes suturing the bowel parts 61 and 63, first bringing together anvil 21 and cartridge 25 by wheel 45 (FIG. 9) and, then, bringing together lever 46 and handle 43 (FIG. 10). After bowel suturing, surgeon withdraws inner unit A from bowel through incision 62, disconnects units bodies connector 32 inside body cavity 58, and withdraws inner unit A from body cavity 58 through hand port 60. All the manipulations in body cavity are executed with the participation of surgeon hand inserted into body cavity through hand port 60.

As applied to thoracal cavity, the operation method provides for forming at least two—the first and the second surgical openings in patient's thoracal cavity: second surgical opening begins below a costal arch in epigastrium and passes into thoracal cavity to provide the access of surgeon's hand into thoracal cavity and designed for inserting therethrough device inner unit into thoracal cavity and for participating the surgeon hand in surgical manipulations inside thoracal cavity; the first surgical opening located in intercostal gap, designed for leading the free end of intermediate part through thoracal cavity wall, and having minimally needed maximal transverse dimension substantially lesser than one of the second surgical opening and the inner unit. Further, the method includes: inserting the inner unit into thoracal cavity through the second surgical opening, leading the free end of intermediate part through body cavity wall via the first surgical opening, and connecting the inner unit to the outer unit by the coupling members with forming an operational assembly. As a result, an assembly inner part is disposed inside thoracal cavity, an assembly outer part is disposed outside thoracal cavity, and the intermediate part is passing through the first surgical opening. After the execution of needed surgical operations by the mentioned operational assembly and surgeon hand disposed inside body cavity, surgeon disconnects the inner unit from the outer unit, withdraws the intermediate part from the first surgical opening and the inner unit from thoracal cavity through the second surgical opening.

In version embodiment, the second surgical opening is passed through abdominal cavity and, further, through patient's diaphragm into thoracal cavity.

In version embodiment, a hand port is mounted into the second surgical opening and inserting the inner unit into thoracal cavity and its holding during its connecting with the outer unit are executed by surgeon's hand inserted into thoracal cavity through the hand port.

In version embodiment, the first surgical opening in intercostal gap of thoracal cavity wall is executed by a trocar device with mounting a trocar port in the first surgical opening.

Thus, only small first surgical opening 56 is necessary for inserting surgical instrument into body cavity 58. Specifically, diameter of intermediate part body 29 determining the diameter of first surgical opening lies in the limits of 6-13 mm. Upon using many surgical instruments, this provides substantial decrease of patient's traumatization. There is a possibility of using the inner units of various dimensions adapted to connection with the same outer unit C and intermediate part B. This allows the decrease of total instrument cost and needed number of first surgical opening. The latter additionally decreases patient's traumatization. The present invention enables the use of the inner units without substantial restrictions their dimensions due to their insertion into body cavity through hand port. As a result, the potentialities of MIS are substantially extended. Moreover, this allows the adapting for MIS various functional heads applied in open surgery thereby reducing the time and investments for R&D. The use of hand port 60 provides free access of surgeon hand into body cavity 58 and its participation in surgical manipulations that significantly improves an operation convenience.

In version embodiment (not shown), the minimally invasive device has an electro-mechanical, or pneumatic actuating means and a drive means as distinct from mechanical actuating means 31 and drive means 30 of the above circular stapler.

In version embodiment (not shown), the second surgical opening is executed by a trocar device, which has internal dimensions allowing the insertion of the inner unit into body cavity through its trocar port. This inserting and subsequent holding the inner unit during its connection with the outer unit are executed by an inner unit holder inserted into body cavity through the mentioned trocar port.

In version embodiment, the inner unit and intermediate part are made as an integral unit, the coupling means are disposed on the intermediate part and on the outer unit and adapted to the connection/disconnection beyond body cavity after inserting the inner part into body cavity through the second surgical opening and leading the intermediate part free end via the first surgical opening from the inside to the outside of body cavity.

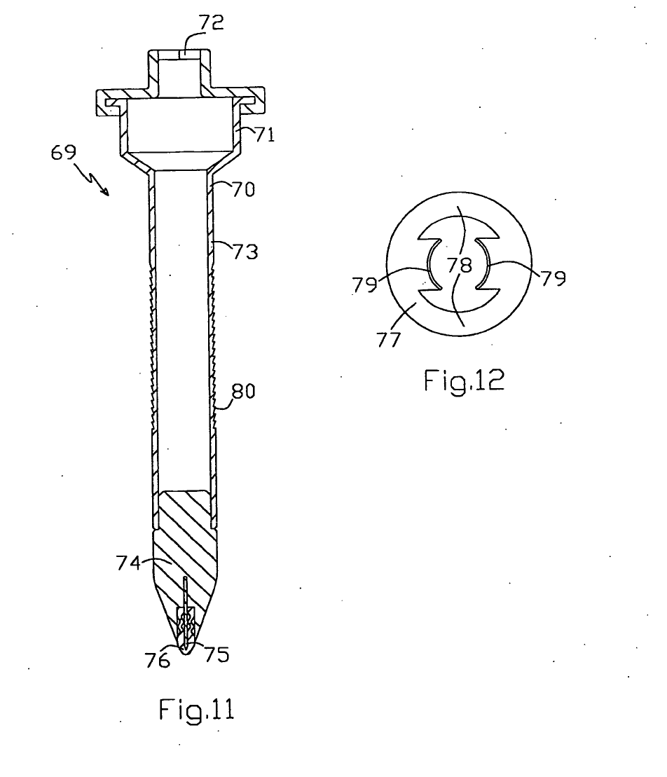

In version embodiment (FIGS. 11-15), the trocar device used for the execution of first surgical opening 56 has inner part 69 in the form of a trocar portal unit including trocar port 70 with inner unit 71 comprising sealing means 72, which presents an elastic washer with a few central slots operating both as a trocar valve and a sealing member. Further, the trocar device includes intermediate part ⁷³ as a part of trocar port 70 and detachable obturatorless penetrating unit 74 having a sharp penetrating knife 75 and resilient protector member 76. There is also the outer unit presenting fixation member 77. Fixation member 77 has resilient sides 78, which, under their compression by surgeon, increase the distance between clamping members 79 thereby allowing the montage of fixation member 77 onto trocar port 70. The latter has indented surface 80 facilitating its engagement with members 79.

In version embodiment (not shown), trocar port 70 has smooth external surface, which engages fixation member 77 due to a clipping means located on fixation member 77.

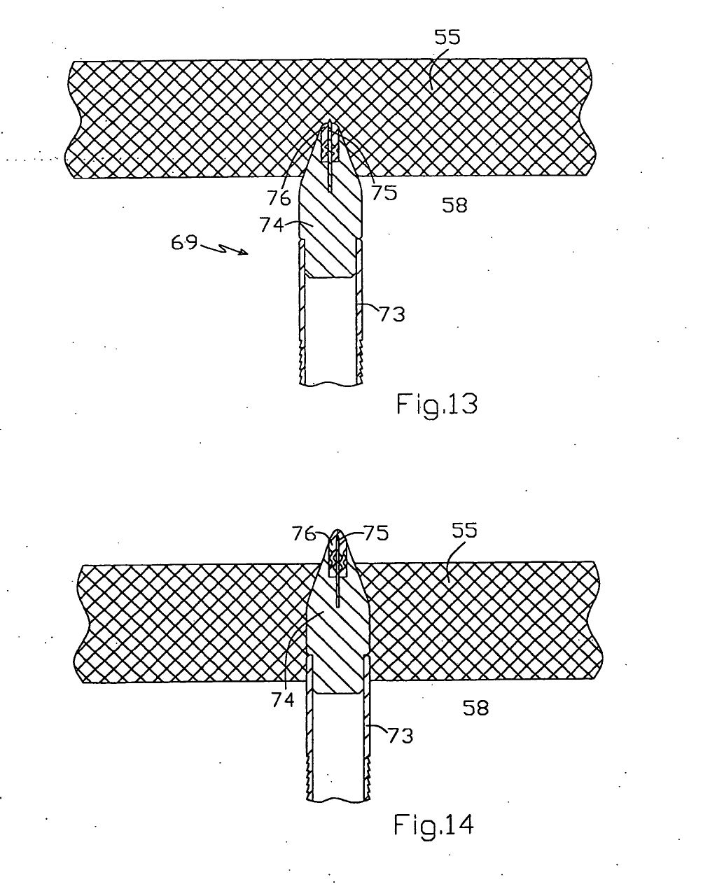

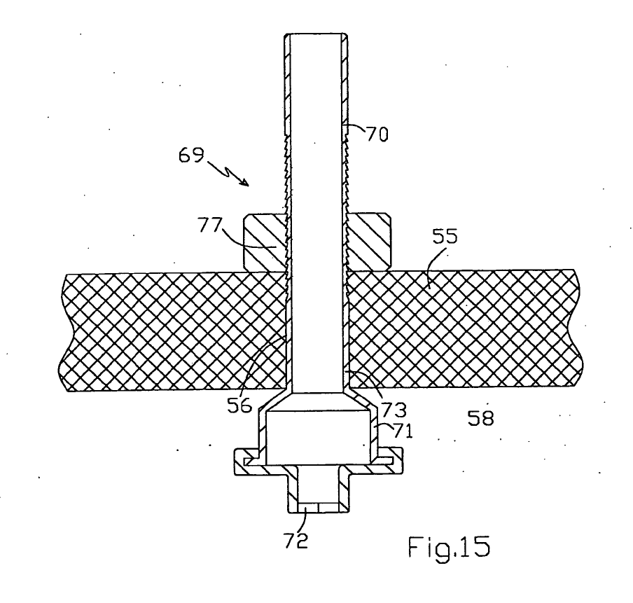

The trocar inner part 69 is inserted into body cavity 58 through the second surgical opening 59, and surgeon pierces body cavity wall 55 by trocar inner part 69 from the inside of body cavity 58 up to the abutment of the inner unit 71 against body cavity wall 55 thereby forming the first surgical opening 56 and simultaneously mounting the trocar port 70 therein. During body cavity wall piercing, resilient protector member 76 is compressed by body's tissue and cut through by knife 75 allowing knife 75 cutting the tissue (FIG. 13). At the moment of penetrating unit going out of body wall (FIG. 14), protector member 76 is returned to its initial position under its elasticity forces thereby protecting knife 75. Then, surgeon removes detachable penetrating unit 74 and mounts fixation member 77 on the exterior end of trocar port 70 thereby securing trocar port 70 in body cavity wall (FIG. 15). This trocar device is simpler, cheaper, and safer in use in comparison with traditional versions having the obturator and inserted from the outside of body cavity.

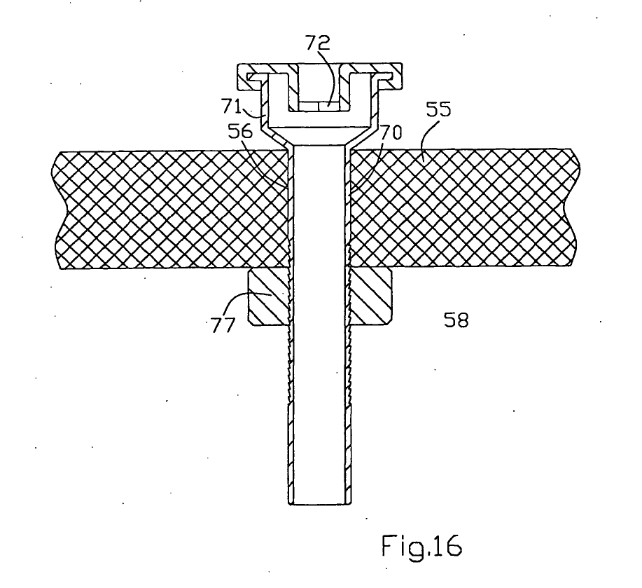

In version embodiment (FIG. 16), the above described trocar device is inserted into body cavity wall from the outside of body cavity. In this case, unit 71 with sealing means 72 presents the device outer unit and fixation member 77 presents the inner unit, which is mounted onto trocar port 70 inside body cavity by surgeon's hand inserted into body cavity through second surgical opening 59 after removing penetrating unit 74 from the trocar port end disposed inside body cavity.

Similar to last version of the use of fixation member 77, this member inserted into body cavity through the second surgical opening can be used as a fixation member of traditional trocar devices by its mounting onto trocar port distal end inside body cavity.

In version embodiment (not shown), the free end of surgical instrument intermediate part is provided with a penetrating tip identical to penetrating unit 74 and designed for piercing body cavity wall to form the first surgical opening and simultaneously to insert the intermediate part into formed first surgical opening. This allows the exclusion of trocar devices from instruments set and surgical procedure thereby substantially lowering set's cost and significantly simplifying and reducing surgical procedure.

In version embodiment (not shown), the body of the inner unit of assembled device is rotatable around the device longitudinal axis and relative to the body of the outer unit thereby providing the property inherent in traditional MIS instrument.

In version embodiment (not shown), the inner unit presents a suturing head of a linear stapler.

In version embodiments, the minimally invasive device according to the present invention is made as: an electrosurgical instrument, or a camera, or a surgical laser, or a medical endoscope. These devices include all the main features of the present invention such as outer unit, inner unit, intermediate part, and coupling means with their mutual arrangement, general designation, and use identical with those of the above described surgical stapler.

The present invention, in principle, allows the use for MIS the instrument set including one hand port, one trocar device, one outer unit, and a plurality of the inner units of various functional designation and transverse dimensions adapted to connecting and using with one outer unit. In another version embodiment, the instrument set includes one hand port, a few (restricted number) trocar devices and outer units of the same dimensions, and a plurality of various inner units needed for the implementation of definite surgical procedure.

Proposed minimal invasive device consisting of detachable inner and outer units is universal instrument allowing its use both in present proposed MIS version and in usual open surgery, as well as in traditional MIS as a combination of traditional miniature functional head (inner unit) and the outer part of proposed device.

1. A minimal invasive device for minimal traumatic surgery partly introduced into body cavity, assembled through at least two—the first and the second surgical openings in a body cavity wall, passing through said first surgical opening during device operation, and having:
An inner unit, an outer unit, and an intermediate part, which is an integral part of one of said units, has a free end, is designed to passing through body cavity wall via said first surgical opening, and having a maximal transverse dimension substantially lesser than a maximal transverse dimension of said inner unit; said intermediate part in assembled operating devise is disposed between said units and inside said first surgical opening,
Said inner unit designed to inserting into body cavity and withdrawing therefrom through said second surgical opening and having maximal transverse dimension, which is substantially more than maximal transverse dimension of said first surgical opening minimally needed for inserting therethrough said intermediate part thereby eliminating the insertion of said inner unit into body cavity and withdrawing therefrom through said first surgical opening,
A coupling means partly disposed on said intermediate part and adapted to operative connecting and disconnecting said inner unit and said outer unit while said inner unit is disposed inside body cavity.

2. Device of claim 1, wherein said maximal transverse dimension of intermediate part does not exceed 13 mm.

3. Device of claim 2, wherein said maximal transverse dimension of intermediate part does not exceed 10 mm.

4. Device of claim 1, wherein said outer unit and said intermediate part are made as an integral outer part and said coupling means are disposed on said intermediate part and on said inner unit and adapted to the connection and disconnection of said outer part and said inner unit in body cavity after inserting said inner unit into body cavity through said second surgical opening and said free end of intermediate part into body cavity through said first surgical opening.

5. Device of claim 1, wherein said inner unit and said intermediate part are made as an integral inner part and said coupling means are disposed on said intermediate part and on said outer unit and adapted to the connection and disconnection of said outer unit and said inner part beyond body cavity after inserting said inner part into body cavity through said second opening and leading said free end of intermediate part through body cavity wall via said first surgical opening from the inside to the outside of body cavity.

6. Device of claim 1, wherein said free end of intermediate part is provided with a penetrating tip designed for piercing body cavity wall to form said first surgical opening and simultaneously to insert said intermediate part into formed said first surgical opening.

7. Device of claim 1, wherein said inner unit has an inner unit body, said outer unit has an outer unit body, there is a transmitting means passing in said intermediate part and designed to the transmission of functional action between said outer and inner units, and said coupling means have a units bodies connector for the connection of said inner unit body to said outer unit body.

8. Device of claim 7, wherein said outer unit has an actuating means, said transmitting means presents a drive means, and said functional action is a control movement of said actuating means transmitted by said drive means to said inner unit.

9. Device of claim 3, wherein said maximal transverse dimension of said intermediate part does not exceed 6 mm.

10. Device of claim 7, wherein said transmission of functional action presents the transmission of electric power.

11. Device of claim 10, presenting an electrosurgical instrument.

12. Device of claim 7, wherein said transmission of functional action presents the transmission of electromagnetic wives.

13. Device of claim 10, presenting a camera.

14. Device of claim 12, which presents a surgical laser.

15. Device of claim 12, which presents a medical endoscope

16. Device of claim 8, wherein in assembled device, said inner unit body is immovable relative to said outer unit body in the direction along a longitudinal axis of said assembled device.

17. Device of claim 16, wherein said coupling means includes a drive means connector.

18. Device of claim 16, wherein said inner unit body in said assembled device is rotatable around said longitudinal axis and relative to said outer unit body.

19. Device of claims 16, presenting a surgical stapler for applying a plurality of staples to patient's tissues, wherein said inner unit has a staples' cartridge and an anvil adapted to juxtaposition with said staples' cartridge and bending said staples to suture patient's tissues.

20. Device of claim 4, 17, 19, presenting a circular stapler for bowel circular anastomosis, wherein said anvil is disposed distally of said staples' cartridge, there is an anvil unit including said anvil and an anvil shaft, there is a cartridge unit including said inner unit body, said cartridge, and a connector lock means eliminating the disconnection of said drive means connector at least during tissue stapling, said cartridge unit is secured to a body of said intermediate part by said units bodies connector, said drive means include at least one control rod connecting said actuating means and said anvil shaft, and said drive means connector includes an anvil shaft engagement member disposed on a proximal free end of said anvil shaft and a control rod engagement member disposed on a distal free end of said control rod.

21. Device of claim 19, presenting a linear stapler.

22. Device of claim 1, wherein said inner unit is adapted to inserting into body cavity through a hand port mounted in said second surgical opening.

23. Device of claim 1, wherein said inner unit is adapted to inserting into body cavity through a trocar port mounted in said second surgical opening.

24. Device of claim 4, presenting a trocar device, wherein said outer part presents a trocar portal unit including a trocar port with a sealing means, said outer unit, said intermediate part, and a penetrating means having a protective means, and said inner unit presents an interior fixation member connected to said trocar port inside body cavity to fix said trocar port in body cavity wall after forming said first surgical opening by means of said penetrating means and leading said trocar port distal end through said first surgical opening from the outside to the inside of body cavity.

25. Device of claim 5, presenting a trocar device, wherein said inner part presents a trocar portal unit including a trocar port with a sealing means, said inner unit, said intermediate part, and a penetrating means having a protective means, and said outer unit presents an exterior fixation member connected to said trocar port on the outside of body cavity to fix said trocar port in body cavity wall after forming said first surgical opening by means of said penetrating means and leading said trocar port distal end through said first surgical opening from the inside to the outside of body cavity.

26. A set of surgical instruments for minimal traumatic surgery executed through at least two—the first and the second surgical openings in a body cavity walls, including at least two—the first and the second ports mounted in said first and second surgical openings, respectively, as well as a minimally invasive device assembled through at least two—said first and said second ports, passing through said first port during device operation, and having:
An inner unit, an outer unit, and an intermediate part, which is an integral part of one of said units, has a free end, is designed to passing its said free end through body cavity wall via said first port, and having a maximal transverse dimension substantially lesser than a maximal transverse dimension of said inner unit; said intermediate part in assembled operating devise is disposed between said units and inside said first port,
Said inner unit designed to inserting into body cavity and withdrawing therefrom through said second port and having maximal transverse dimension, which is substantially more than maximal transverse inner dimension of said first port minimally needed for inserting therethrough said intermediate part thereby eliminating the insertion of said inner unit into body cavity and withdrawing therefrom through said first port,
A coupling means disposed on said intermediate part and on one of said units and adapted to operative connecting and disconnecting said inner unit and said outer unit while said inner unit is disposed inside body cavity.

27. Set of claim 26, wherein said outer unit and said intermediate part are made as an integral outer part and said coupling means are disposed on said intermediate part and on said inner unit and adapted to the connection and disconnection of said outer part and said inner unit in body cavity after inserting said inner unit into body cavity through said second port and said free end of intermediate part into body cavity through said first port.

28. Set of claim 26, wherein said inner unit and said intermediate part are made as an integral inner part and said coupling means are disposed on said intermediate part and on said outer unit and adapted to the connection and disconnection of said outer unit and said inner part beyond body cavity after inserting said inner part into body cavity through said second port and leading said free end of intermediate part through said first port from the inside to the outside of body cavity.

29. Set of claim 26, wherein said inner unit has an inner unit body, said outer unit has an outer unit body, there is a transmitting means passing in said intermediate part and designed to the transmission of functional action between said outer and inner units, and said coupling means have a units bodies connector for the connection of said inner unit body to said outer unit body.

30. Set of claim 26, wherein said second port presents a hand port.

31. Set of claim 26, wherein an internal diameter of said first port does not exceed 13 mm.

32. Set of claim 26, including at least one trocar device for the execution of at least one said first surgical opening and mounting at least one first port therein.

33. Set of claims 28, 32, wherein said trocar device has said inner part presenting a trocar portal unit including a trocar port with a penetrating means and said outer unit presenting an exterior fixating member connected to said trocar port outside body cavity to fixate said trocar port in body cavity wall after leading its distal end through said first surgical opening from the inside to the outside of body cavity.

34. Set of claim 33, wherein there are a sealing means including a sealing member and a trocar valve disposed on said trocar port mounted in body cavity wall.

35. Set of claim 33, wherein said penetrating means presents an obturatorless unit detachably connected to a distal end of said trocar port and having a penetrating sharp tip provided with a protective means for its preventing from any contact with staff.

36. Set of claims 30, 32, including one said hand port and at least one said trocar device used for execution of a few said first surgical openings and mounting said first ports of equal dimensions therein.

37. Set of claims 30, 32, including one said hand port, at least one said trocar device, and a plurality of said inner units of various functional designations and transverse dimensions adapted to connecting and using with at least one said outer unit.

38. Method of preparation and execution of minimally traumatic surgical operations implemented with at least one minimally invasive device comprising an inner unit, an outer unit, and an intermediate part, which is an integral part of one of said units, has a free end, is designed to passing its said free end through body cavity wall, and having a maximal transverse dimension substantially lesser than a maximal transverse dimension of said inner unit, and coupling members disposed on said intermediate part and on one of said units and adapted to operative connecting and disconnecting said inner and outer units,
said method comprising:
Forming at least two—the first and the second surgical openings in body cavity walls: said second surgical opening is designed for inserting said inner unit therethrough into body cavity and said first surgical opening is designed for leading said free end of intermediate part through body cavity wall and having minimally needed maximal transverse dimension substantially lesser than one of said second surgical opening and said inner unit,
Inserting said inner unit into body cavity through said second surgical opening,
Leading said free end of intermediate part through body cavity wall via said first surgical opening,
Connecting said inner unit to said outer unit by said coupling members with forming an operational assembly so that an assembly inner part is disposed inside body cavity, an assembly outer part is disposed outside body cavity, and said intermediate part is passing through said first surgical opening,
Execution of surgical operations at least by means of said operational assembly,
Disconnecting said inner unit from said outer unit after executing needed surgical operations, withdrawing said intermediate part from said first surgical opening, and withdrawing said inner unit from body cavity through said second surgical opening, thus, the components of said minimally invasive device are removed from body cavity trough the same openings through which they were inserted.

39. Method of claim 38, wherein a hand port is mounted in said second surgical opening and said inserting said inner unit into body cavity and its holding during said its connecting with outer unit are executed by surgeon's hand inserted into body cavity through said hand port.

40. Method of claim 38, wherein said second surgical opening is executed by a trocar device with mounting a trocar port having internal dimensions allowing said inserting said inner unit into body cavity, therewith said inserting said inner unit into body cavity and its holding during said its connecting with outer unit are executed by an inner unit holder inserted into body cavity through said trocar port.

41. Method of claim 38, wherein at least one said first surgical opening is executed by a trocar device with mounting a trocar port in at least one said first surgical opening.

42. Method of claim 41, wherein, first, at least part of said trocar device is inserted into body cavity through said second surgical opening and, then, it is used for piercing the body cavity wall from the inside of body cavity for said execution of said first surgical opening and mounting therein said trocar port.

43. Method of claim 42, implemented with said trocar device having said inner part in the form of a trocar portal unit including a trocar port with sealing means, said inner unit, said intermediate part, and a detachable obturatorless penetrating unit with a sharp penetrating end and a protective means, and said outer unit presenting an exterior fixating member; (method) wherein, first, said trocar device inner part is inserted into body cavity through said second surgical opening, further, surgeon, acting via said second surgical opening, pierces body cavity wall by said trocar device inner part from the inside of body cavity up to the abutment of said inner unit against body cavity wall to form said first surgical opening and mount said trocar port therein, then surgeon removes said detachable penetrating unit from an exterior end of said trocar port and mounts said exterior fixating member thereon to secure said trocar port in body cavity wall.

44. Method of claim 38, implemented with said minimally invasive device having said intermediate part, whose said free end is provided with a sharp penetrating tip; (method) wherein said forming said first surgical opening and said inserting said intermediate part into first surgical opening is executed simultaneously as a result of through piercing the body cavity wall by said intermediate part with sharp penetrating tip, thereafter, surgeon executes said connecting said inner and outer units and said forming said operational assembly.

45. Method of claims 38, wherein one said second surgical opening and a few said first surgical openings are executed and used.

46. Method of claims 38, wherein one said second surgical opening, at least one said first surgical opening, and a plurality of said inner units of various designations and transverse dimensions are used, therewith said various inner units are inserted into body cavity through said second surgical opening for connecting to at least the same said outer unit.

47. Method of preparation and execution of thoracic minimally traumatic surgery implemented with at least one minimally invasive device comprising an inner unit, an outer unit, an intermediate part, which is an integral part of one of said units, has a free end, is designed to leading its said free end through body cavity wall, and having a maximal transverse dimension substantially lesser than a maximal transverse dimension of said inner unit; and coupling members disposed on said intermediate part and on one of said units and adapted to operative connecting and disconnecting said inner and outer units,
said method comprising:
Forming at least two—the first and the second surgical openings in patient's thoracal cavity: said second surgical opening begins below a costal arch and passes into thoracal cavity to provide the access of surgeon's hand into thoracal cavity and designed for inserting therethrough said inner unit into thoracal cavity; said at least one first surgical opening located in intercostal gap, designed for inserting said free end of intermediate part through thoracal cavity wall, and having minimally needed maximal transverse dimension substantially lesser than one of said second surgical opening and said inner unit,
Inserting said inner unit into thoracal cavity through said second surgical opening,
leading said free end of intermediate part through body cavity wall via said first surgical opening,
Connecting said inner unit to said outer unit by said coupling members with forming an operational assembly so that an assembly inner part is disposed inside thoracal cavity, an assembly outer part is disposed outside thoracal cavity, and said intermediate part is passing through said first surgical opening,
Execution of surgical operations at least by said operational assembly,
Disconnecting said inner unit from said outer unit after executing needed surgical operations, withdrawing said intermediate part from said first surgical opening, and withdrawing said inner unit from thoracal cavity through said second surgical opening by surgeon's hand.

48. Method of claim 47, wherein said second surgical opening is passed through abdominal cavity and further through patient's diaphragm.

49. Method of claims 47, wherein a hand port is mounted into said second surgical opening and said inserting said inner unit into thoracal cavity and its holding during said its connecting with outer unit are executed by surgeon's hand inserted into thoracal cavity through said hand port.

50. Method of claim 47, wherein at least one said first surgical opening is executed by a trocar device with mounting a trocar port in at least one said first surgical opening.

51. Method of claim 47, wherein surgeon helps to execute the surgical operations by his hand inserted into thoracal cavity through said second surgical opening.

52. Method of preparation and execution of thoracic minimally traumatic surgery implemented with at least one minimally invasive device having a functional head and an actuating means,
said method comprising:
Forming at least two—the first and the second surgical openings in patient's thoracal cavity: said second surgical opening begins below a costal arch, passes into thoracal cavity and designed for inserting surgeon's hand into thoracal cavity, and at least one said first surgical opening located in intercostal gap, designed for the operation of said minimally invasive device in thoracal cavity, and having minimally needed maximal transverse dimension substantially lesser than one of said second surgical opening,
Inserting surgeon's hand into thoracal cavity through said second surgical opening,
Inserting said minimally invasive device into thoracal cavity at least partly through said first surgical opening so that its said functional head is disposed in thoracal cavity and its said actuating means is disposed outside thoracal cavity,
Execution of surgical operations by said at least one minimally invasive device and surgeon hand inserted into thoracal cavity through said second opening.

53. Method of claims 52, wherein a hand port is mounted into said second surgical opening.
